(19) United States
(12) Patent Application Publication
(10) Pub. No.: US 2003/0165529 A1
(43) Pub. Date: Sep. 4, 2003

(54) Vaccine against tyrpanosomiasis

(76) Inventors: Roger K. Prichard, Beaconsfield (CA); George W Lubega, Kampala (UG); Denis K Byarugaba, Kampala (UG); Donosian O K Ochola, Kampala (UG)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/203,487

(22) PCT Filed: Feb. 7, 2001
(86) PCT No.: PCT/CA01/00140

(51) Int. Cl. A61K 39/002; A61K 38/16; C07K 14/44; C07K 16/20

(52) U.S. Cl. 424/191.1; 530/388.6; 530/358

(57) ABSTRACT

The present invention relates to the immunization of animals and humans with trypanosome tubulin to protect against trypanosomes. More particularly, the present invention relates to a substantially pure tubulin preparation, which comprises a tubulin extract from Trypanosoma brucei which tubulin preparation can protect animals and humans against heterologous strains of different species of Trypanosoma.

### BACKGROUND OF THE INVENTION

(a) Field of the Invention

The invention relates to the immunization of animals and humans with trypanosome tubulin to protect against trypanosomes.

(b) Description of Prior Art

African trypanosomes belong to the genus Trypanosoma, are transmitted by tsetse flies of the genus Glossina and cause a variety of severe diseases collectively known as trypanosomiasis in man and animals. The main pathogenic species in animals are T. conglense, T. vivax, T. smiae and T. b. brucei and the disease they cause is collectively known as Nagana. T. b. brucei is morphologically indistinguishable from the human parasites, T b. gambiense and T. b. rhodesienise which, respectively, cause the chronic Gambian and the acute Rhodesian types of sleeping sickness. However, T. b. brucei cannot infect humans and becomes lysed in human blood in vitro, but under certain conditions can switch to a human form and vice versa. Therefore, the current control methods and the search for novel control tools for the human and animal diseases are linked.

Immunological control has been frustrated by antigenic variation (Barry, J. D. (1997) Parasitology Today, 13:212-217) and as such the control of African trypanosomiasis is restricted to vector control, chemoprophylaxis (in animals) and treatment of sick animals and humans (Barret, J. C. (1997) Control Strategies of African Trypanosomiases: Their sustainability and Effectiveness. In: G. Hides, J. C. Mottram, G. H. Combs and P. H Holmes, (Eds.) Trypanosomiasis and Leishhmaniasis. pp 347-362). Each of these approaches has important limitations (Barret, J. C. (1997) Control Strategies of African Trypanosomiases: Their sustainability and Effectiveness. In: G. Hides, J. C. Mottram, G. H. Combs and P. H Holmes, (Eds.) Trypanosomiasis and Leishmaniasis. pp 347-362) and the search for new drugs continues at a low level. However, typanosomiasis remains a major tropical disease, affecting mainly the poor of the world who don't attract the interest of the pharmaceutical companies.

Trypanosomes have a unique capacity for antigenic variation at the cell surface which is the basis of their ability to evade the host immune response and because of this, prospects for the development of a vaccine against African trypanosomiasis have been considered poor.

Despite the poor prospects of finding a vaccine, the most effective and sustainable way of controlling trypanosomiasis would be a safe and cost-effective vaccine (Newman, M. J. et al. (1995) Immunological Formulation design considerations for subunit vaccines. In: M. F. Powell and M. J. Newman (ed) The Subunit and Adjuvant Approach. Plenum Press, New York). Because of this, the search for a suitable anti-trypanosome vaccine continues.

Partial protection has been reported against African trypanosomosis using irradiated trypanosomes (Morrison, W. I. et al. (1982) Parasite Immunology, 4: 395-407). The infection and treatment method has also been tried but only increased the prepatent period and survival time after challenge but did not prevent infection (Scott, J. M. et al. (1978) Reviews in Veterinary Science, 25: 115-117).

Surface antigens that can be used as the basis for a vaccine against trypanosomiasis include, two glycoproteins namely, the variable surface glycoprotein (VSG) of the bloodstream forms, which occur in mammalian hosts, and procyclin of the procyclics which occur in the-insect vectors. Procyclin has not attracted a lot of attention for vaccine development. The mammalian host makes a good immune response to the first wave of invading trypanosomes by producing antibodies against the first wave of VSGs called metacyclic variable antigenic types (M-VATs). However before all the parasites can be eliminated, trypanosomes switch to the blood stream VSGs which also induce protective antibodies but again before these are removed by this new wave of antibodies, more trypanosomes (approximately one in every 10⁴-10⁵) turn off the genes controlling the expression of the initial VSG and switch to genes for expression a different VSG molecule, not recognized by the animal's initial immune response; and so the process continues with the parasite population bearing new VSGs and always keeping a step ahead of the host's immune system (Barry, J. D. (1997) Parasitology Today, 13:212-217). Protective immunity can readily be achieved against a given trypanosome strain but because of antigenic variation animals remain susceptible to heterologous challenge (Scott et al., 1978). Trypanosomes are able to express an infinite variety of VSGs and therefore a vaccine based on these abundant surface antigens is now out of the question.

Given their functional role, plasma membrane proteins are unlikely to undergo antigenic variation (Barry, J. D. (1997) Parasitology Today, 13:212-217) in the same manner as the VSGs and consequently may represent suitable targets for the development of vaccines. However, since they are concealed by the glycoprotein envelope, studies with the plasma membrane proteins have not yielded encouraging results (Murray, M. et al. (1985) Parasitology, 91:53-66).

Cytosolic fractions of trypanosomes have also been used and shown to have constant antigenic properties though they have been considered poor immunogens. The use of purified antigens has an advantage in that it doesn't include irrelevant antigens or proteins which may overwhelm or suppress the host immune system. In addition, knowing a specific immuno-protective antigen would enable cloning and synthesis of its recombinant form and its known DNA sequence would thus allow further modifications to optimize immuno-protection attributes. However, pure trypanosome proteins as such have not been identified for vaccination, but flagella pocket fractions of African trypanosomes have been used to immunize laboratory or large animals (Mkunza, F. et al. (1995) Vaccine, 13: 151-154). These studies showed that susceptible hosts may be partially protected whereby the immunized animals lived longer than the controls upon challenge with a lethal dose of trypanosomes. However, studies resulting in complete protection have also been reported using the flagella fraction quiz, A. M. et al. (1990) Molecular and Biochemical Parasitology, 39: 117-126) of T. cruzi and a fraction of T. brucei consisting of a microtubule associated protein (MAP 52) and two glycosomal enzymes (Balaban, N. et al. (1995) Journal of Infectious Diseases, 172: 845-850). On the other hand, immunization with similar flagella pocket fractions of T. rhodesiense in mice and cattle (Mkunza F. et al. (1995) Vaccine, 13: 151-154) gave a partial protection to heterologous challenge. Certain recombinant antigens have also been explored for protection against T. cruzi infection (Taibi, A. et al. (1995) Immunology Letters, 48: 193-200; Costa, F. et al. (1998) Vaccine 16: 768-774; Wizel, B. and Tarleton, R L. (1998) Infection and Immunity, 66; 5073-5081) but their application in African trypanosomiasis has not been reported.

Our interest has been focused on the cytoskeleton and particularly the microtubules (Lubega, G. W. et al. (1998) South African Journal of Science. 94 284-285). The cell body of trypanosomes is tightly enveloped by a compact single layer of microtubules, which are situated immediately beneath the surface membrane. These pellicular microtubules provide a high degree of flexibility to the cells, mechanical stability and motility and together form the dominant cellular architecture. Microtubules are also found in the flagellum, where they form one of the two prominent structures of this organelle, the axoneme. The other is the paraxial rod which is essentially a network of actin fibers, which extends along the axoneme and stays in close contact with it. In trypanosomes, it has been observed that pellicular and flagella microtubules are immunologically distinct. A third domain of microtubule function in the trypanosome is the formation of the spindle apparatus of dividing nuclei. Microtubules are cross linked to the plasma membrane by MAPs and together build into complex assemblies such as the mitotic spindle, flagella, axonemes and neurotubules.

The major building block of microtubules is a protein known as tubulin which is usually a heterodimer of α and β subunits and exists in all eukaryotic cells. However, the properties of tubulin of lower eukaryotes such as protozoa and helminths differ from those of higher ones such as mammals which makes it possible to selectively target the parasite tubulin. Consequently, tubulin is the target of benzimidazole anthelmintics.

Tubulins are a multigene family of related proteins which, in trypanosomes, are comprised of three related proteins each about 55 kDa termed α, β and γ-tubulin (Kimmel, B. et al. (1985) Gene, 35: 237-248). Whereas α-tubulin of trypanosomes exists in two isoforms α1- and α3-tubulin, β-tubulin has only one single isoform which is very interesting because the β-tubulin appears to be the primary target for chemotherapy (Lubega, G. W. and Prichard, R. K (1991) Haemonchus cointortus. Molecular and Biochemical Parasitology, 47: 129-138) and probably for immunotherapy (Lubega, G. W. et al. (1998) South African Journal of Science. 94 284-285).

In this study, trypanosome tubulin was investigated for its potential as a vaccine target. The rationale for using tubulin was that it participates in very vital cellular functions, it is well distributed in the trypanosomes, there are differences between the mammalian and trypanosome tubulin and its biochemical nature remains unchanged throughout the life cycle of the trypanosomes and it is the single most abundant protein of the cytoskeleton.

The potential of tubulin as an immunotherapeutic target was demonstrated with Brugia pahangi whereby monoclonal antibodies raised against β-tubulin peptides destroyed the surfaces of the filarail worms in vitro and reduced microfilaraemia and the survival of the adult worms in vivo (Bughio, N. I. et al. (1993) International Journal of Parasitology, 23: 913-924).

Lubega et al. (Lubega, G. W. et al. (1998) South African Journal of Science. 94 284-285) showed that the tubulin enriched extract from a strain of T brucei conferred protection against the same strain of T. brucei in vivo, or inhibited the development of the same strain of T. brucei in vitro. Thus there was evidence for protection against homologous strain of Trypanosoma brucei. Homologous protection has been acheived before with other trypanosome extracts and it is known that variable surface glycoprotein (VSGP) of trypanosomes can confer very strong homologous protection. However, because in vivo, the trypanosome can change its VSGP, once the host mounts a strong immune response, the parasite can continue to proliferate and attempts to develop a vaccine against sleeping sickness and Nagana have so far been frustrated.

It would be highly desirable to be provided with means for the immunization of animals and humans with trypanosome tubulin to protect against any trypanosomes or to provide for protection against heterologous strains of different species of Trypanosoma.

### SUMMARY OF THE INVENTION

One aim of the present invention is to provide means for the immunization of animals and humans with trypanosome tubulin to protect against any trypanosomes or to provide for protection against heterologous strains of different species of Trypanosoma.

Surprisingly and in accordance with the present invention, there is provided a purified tubulin preparation from trypanosome which can produce a strong protection not only against the homologous strain (from which the tubulin antigen was prepared), but also against heterologous strains of different species of Trypanosoma. Furthermore, the data of the present invention demonstrate that the protection is independent of VSGP.

In accordance with the present invention, there is provided a substantially pure tubulin preparation, which comprises a tubulin extract from Trypanosoma brucei, wherein said tubulin preparation can protect animals and humans against heterologous strains of different species of Trypanosoma.

In accordance with another embodiment of the present invention, there is provided a method for the immunization of an animal or a human patient against heterologous strains of different species of Trypanosoma, which comprises administering to said animal or human patient an immunogenic amount of a tubulin extract preparation isolated from a Trypanosoma.

The preferred Trypanosoma is Trypanosoma brucei.

In accordance with another embodiment of the present invention, there is provided a vaccine against trypanosomiasis in animals or humans, which comprises an immunogenic amount of a tubulin extract preparation isolated from a Trypanosoma or an immunoprotective amount of an antibody raised against a tubulin isolated from a Trypanosoma.

In accordance with another embodiment of the present invention, there is provided an antibody raised against the tubulin preparation of the present invention.

The antibody may be a polyclonal or a monclonal antibody.

In accordance with another embodiment of the present invention, there is provided a vaccine against trypanosomiasis in animals or humans, which comprises an immunogenic amount of a recombinant tubulin which corresponds in composition to a tubulin extract preparation isolated from a Trypanosoma or an immunoprotective amount of an antibody raised against said recombinant tubulin.

In accordance with another embodiment of the present invention, there is provided a vaccine against trypanosomiasis in animals or humans, which comprises an immunogenic amount of a synthetic peptide which corresponds in composition to portion of an amino acid sequence of a tubulin extracted from a Trypanosoma or an immunoprotective amount of an antibody raised against said tubulin peptide.

### BRIEF DESCRIPTION OF THE DRAWINGS

FIG. 1 illustrates the purity by SDS-PAGE and Western blot of tubulin purified from T. brucei. Tubulin was purified from T. brucei (T.b) or rat brain (Rb) as described in Materials and Methods and and a sample run on a 10% SDS-PAGE gel and stained with Coomassie blue (Panel A) or processed for Western blot (Panel B) using anti-chicken tubulin monoclonal antibodies before being used in immunization experiments.

FIG. 2 illustrates the effect of the route of immunization or synthetic tubulin peptides (STP) on the rate of antibody development. Mice were immunized with nThTub subcutaneously (sc) or intra-peritoneally (ip), or with STP14 (sc) and booster doses given on day 15 and day 30. The immune or pre-immunization serum were diluted (1:200) and used in ELISA assay. The mean±SD for the pre-immune OD readings was determined and the immune net OD readings calculated by subtracting this mean±2SD from the various immune readings. The results were then plotted as mean net OD readings±SEM (n=10).

FIG. 3 illustrates the specificity by Western blot of the various anti-tubulin antibodies. Trypanosome total soluble extracts from T. brucei UTRO 010291B (T.b), T. rhodesiense UTRO 080291B (T.r) or T. congolense UTRO 161098B (T.c) or rat brain soluble extracts (Rb) were run on a 10% SDS-PAGE and stained with Coomassie blue (Panel A) or transferred to nitrocellulose membrane and probed with various mouse anti-sera as follows: anti-RbTub (Panel B), anti-nThTub (Panel C), anti-dThTub (Panel D) or anti-STT14 (Panel E) or pre-immunization sera (Panel F).

FIG. 4 illustrates the comparison of peak levels and specificity of antibody responses following immunization with the various antigens. Mice were immunized with nThTub, dThTub, ST? 14 or RbTub as described in Materials and Methods. The immune or pre-immunization sera from the mice were diluted (1:200) and cross-tested by poly-L-lysine ELISA against these various antigens.

Net OD readings±SEM (n-10) were calculated as in FIG. 2 above.

FIG. 5 illustrates the effect of dilution and incubation time on trypanosome growth. Trypanosomes were cultured in the presence of different dilutions (x36, x108, x324, x 972, or x 2916) of anti-nThTub immune serun or pre-immunization serum diluted 12 times. Incubation was continued for 8 days with change of medium every 48 hrs. Trypanosome counts were made every 24 hrs using the Improved Neubeur Haemocytometer and counts expressed as cells per 100 ml of incubation medium.

FIG. 6 illustrates the comparison of trypanosome growth inhibition (%) by the various immune sera. T. brucei grown and adapted for continuous growth in complete bloodstream-form medium (CBM) were incubated in the presence of different dilutions of immune sera to native (nTbTub) or denatured (dThTub) T. brucei tubulin, or synthetic tubulin peptides (STP12), and trypanosome counts were made every 24 hrs of incubation. Medium was changed after 48 hrs as described in Materials and Methods. The differences in cell counts, after 24 hrs or 96 hrs, between the control and each test serum were expressed as the percentage of counts in the control. The values are mean±SEM of 4 duplicate experiments.

FIG. 7 illustrates the agglutination of trypanosomes cultured in the presence of immune serum. Trypanosomes in log phase of growth were incubated with pre-immunization serum (A) or anti-nTbTub immune serum (B). Agglutination is evident in (B) at various stages of agglutination (arrows). Free trypanosomes in (B) are deformed but those in (A) are not.

FIG. 8 illustrates the immunofluorescence staining of Trypanosoma brucei. Intact (A) or permeablised (B-F) cells of T. brucei were incubated with immune sera to nThTub (B), dThTub (C), STP12 (D), RbTub (E) or pre-immunization serum (F) and developed with fluorescein-conjugated Protein A as described in Materials and Methods. The uniformly fluorescing permeablised trypanosomes (arrows) in B, C, and D can be seen. The intact (i.e non-permeablised) trypanosomes fluoresced (only spots can be seen) at the posterior (possibly flagella pocket) region (A). The trypanosomes (arrows) in Anti-RbTub (E) or pre-immunization serum (1) did not fluorescence at all. Abbreviations: nThTub=native T. brucei tubulin; dThTub=denatured T. brucei tubulin; STP12=synthetic tubulin peptide 12; RbTub=rat brain tubulin.

FIG. 9 illustrates the neutralization of the trypanosome inhibitory activity of anti-Trypanosome tubulin (anti-NTP) or anti-tubulin peptide (anti-STP) immune sera by SDS-PAGE purified trypanosome tubulin (dNTP). Trypanosomes were cultured in the presence of pre-immune serum (control), or anti-NTP serum pre-incubated with dNTP (dNTP-T), or anti-NTP serum not pre-incubated with dNTP (dNTP-UT), or anti-STP serum pre-incubated with dNTP (dNTP-T) or anti-STP serum not pre-incubated with dNTP (dNTP-UT). Trypanosome cell counts were made after 24 hrs using an improved Neuber hemocytometer and expressed as cells per 100 μl of incubation medium.

### DETAILED DESCRIPTION OF THE INVENTION

Trypanosoma brucei tubulin was purified in its native state or after SDS-PAGE (denatured tubulin) and used to immunize mice or rabbits. Synthetic tubulin peptides (STP) and rat brain tubulin were also used. Immunized mice were challenged with the homologous or heterologous strains of T. brucei or T. congoletise or T. rhodesiense. The rabbit immune sera were used for in vitro trypanosome inhibition studies. Native T. brucei tubulin (nThTub) induced protection in all mice tested of which 60-80% (n=81) were complete protection (mice never became patent) and the remainder partial protection (mice became patent but lived longer than the controls). Not only did the nThTub protect against the homologous strain of T. brucei, but it it evoked an equally effective protection against heterologous strains of T. brucei, T. congolense and T. rhodesietise. However, the denatured T. brucei tubulin (dThTub) or synthetic tubulin peptides (STP) did not protect mice against trypanosome challenge, although the rabbit anti-dThTub or anti-STP sera did inhibit trypanosome growth in culture, but to a lesser extent than the anti-nThTub. The rat brain tubulin (RbTub) did not protect mice against trypanosome challenge, nor did the rabbit anti-RbTub serum inhibit trypanosome growth in culture. The levels and specificity of the induced antibodies were investigated by ELISA and Western blot. nThTub immunization by the subcutaneous route and the intraperitoneal route produced similar high levels of protection and antibody titres. dThTub and STP induced lower levels of antibody response than the nThTub. In Western blots the anti-nThTub, anti-dThTub and anti-STP antibodies recognized the tubulin in extracts from different trypanosome species or strains but not mammalian or chicken tubulin whereas antibodies raised against rat brain tubulin recognised trypanosome and vertebrate tubulin. Of five mice passively given immune sera from a group of mice immunized with nThTub at the minimal effective dose, four were protected while one became patent and died, but lived longer than the controls. This suggests that the protection observed may be humoral. The denatured tubulin and synthetic peptides failed to protect mice; probably because they were less immunogenic (produced much lower antibody response) than the native tubulin. The failure of the rat brain tubulin (RbTub) to cause immunoprotection in mice or the failure of the rabbit anti-RbTub sera to inhibit trypanosomes in culture suggests that the protection is parasite specific and is unlikely to cause an autoimmune reaction. An immunofluorescence test showed that the intact trypanosomes in vitro were not stained (except at a small spot in the flagella pocket region) by any of these antibodies but those that had been permeablised with Triton™-100, were specifically and uniformly labelled by the anti-trypanosome tubulin antibodies. The lack of specific immunofluorescence staining of the trypanosome surface suggests that the variant surface glycoproteins (VSG) did not take part in the immunoprotection. Overall these data suggest that tubulin is a novel and very promising target for the development of a parasite specific, broad spectrum anti-trypanosomiasis vaccine.

Materials and Methods

Animals and Trypanosome Stocks

Swiss mice and white giant rats were obtained courtesy of the Uganda Virus Research Institute, Entebbe and were provided with food and water ad libitum. New Zealand white rabbits (about 8 weeks old) were purchased locally and similarly fed. Trypanosome stocks were kindly provided by Dr. J. C. Enyarn of the Livestock Research Institute (formerly UTRO) Tororo, Uganda and included: two T. brucei stocks (UTRO 010291B and 220291D), one T. rhodesiense stock (UTRO 080291B) and one T. congolense (UTRO 161098B) stock. These stocks were maintained in liquid nitrogen or were propagated in mice or rats.

Harvesting of Trypanosomes for Tubulin Purification

Infected blood from liquid nitrogen was intraperitonealy inoculated into rats. Infection was confirmed by examination of tail blood and parasitaemia estimated by the Marching Method. Blood was collected from those rats with high parasitaemia (about 107/ml) and trypanosomes harvested from it by DEAE 52-cellulose (Sigma) anion exchange. The eluted trypanosomes were pelleted by centrifugation for 10 min at 3,000 g at 4° C. and washed twice by suspension in PEM buffer (100 mM pipes, 1 mM EGTA, 1 mM MgSO₄, 1 mM PMSF, pH 6.9) followed by re-centrifugation as described above. The harvested trypanosomes were stored in liquid Nitrogen until needed for tubulin purification.

Tubulin Purification

Tubulin was purified from one strain of T. brucei (UTRO 010291B). Trypanosomes were mixed with 106 μm glass beads (Sigma) and disrupted for 15 min on ice with a pestle and mortar. The homogenate was then suspended in PEM buffer and centrifuged for 10 min at 3,000 g, 4° C. to pellet the beads and any undisrupted cells. The pellet was then re-homogenized to disrupt any remaining trypanosomes and the above procedure repeated twice to ensure that most cells were disrupted. The various homogenate fractions were pooled and centrifuged for 10 min at 10,000 g, 4° C. to remove the insoluble debris which were discarded and the supernatant further centrifuged for 1 h at 100,000 g, 4° C. The resulting supernatant was incubated for 1 h at 37° C. in the presence of 2 μ/ml taxol to promote tubulin polymerization and then centrifuged for 1 h, at 100,000 g, 25° C. The pellet containing the tubulin polymer was then solubilized in urea and renatured as described below.

Solubilization and Naturation of Tubulin

The tubulin, purified as above, was solubilised as previously described (Lubega, G. W. et al. (1993) Molecular and Biochemical Parasitology, 62: 281-292) of a described procedure. Briefly the pellet was dissolved in 3 ml 8 M urea and incubated at 25° C. for 1 hr and then diluted about 20 times with alkaline buffer pH 10.7 (50 mM KH₂ PO₄, 0.1 mM PMSF, 1 mM EDTA and 50 mM NaCl) and incubated for a further 30 min. The pH was then adjusted to 8.0 and the supernatant concentrated to one third by ultrafiltration in CF50A membrane cones (Amicon) and re-diluted 3 times with MES buffer (0.025 MES, 1 mM EGTA, 0.5 mM MgSO₄, 1 mM GTP, pH 6.0). This was again concentrated to one third and rediluted in MES buffer as described above and this was repeated twice. The final volume was centrifuged for 2 h at 40,000 g, 4° C. to ensure that there was no aggregated tubulin. The purified, renatured tubulin (hereafter referred to as native tubulin) was then stored in liquid nitrogen until needed for immunization and related studies.

Purification of Mammalian (Rat-Brain) Tubulin

The purification of tubulin from rat brain was done by the temperature dependent polymerization and depolymerization method (Shelanski, M. L. et al. (1973) Proceedings of the National Academy of Science of the United States of America, 70: 765-768).

Determination of Tubulin Concentration

Tubulin concentration was determined by the BioRad dye method using bovine serum albumin as standard.

Analysis of the Tubulin Purity and Identify

To estimate the purity of the tubulin to be used in immunizations, a solubilized sample of native tubulin was run on a 10% polyacrylamide gel utilizing a BioRad Mini-protean II electrophoresis cell as described (Lubega, G. W. and Prichard, R. K. (1991) Haemonchus contortus. Molecular and Biochemical Parasitology, 47: 129-138). The gel was processed for Western blot or stained with Coomassie blue and dried using a gel drier (1BioRad) and photodocumented using a MP4 camera system (Sigma).

For Western blot, the protein was transferred to a nitrocellulose membrane (BioRad) using the Mini-Transblot system and protocol (BioRad). The Western blot was performed as described (Lubega, G. W. and Prichard, R. K (1991) Haemooizcus contortus. Molecular and Biochemical Parasitology, 47: 129-138) using mouse anti-chicken tubulin monoclonal antibody (Amersham) and peroxidase-conjugated anti-mouse IgG (Jacksons Immuno research laboratories Inc, Canada). The substrate was 1.3 μM diaminobenzidine containing 0.02% (v/v) H₂O₂.

Recovery of Tubulin From the SDS-PAGE Gel for Immunization

To increase the purity of tubulin samples, tubulin bands were recovered from the SDS-PAGE gel. After SDS-PAGE, the tubulin band was identified using guide strips that were cut from both sides of the gel and stained. The piece of gel containing the tubulin band was sliced out and homogenised in PBS buffer using a polytron homogenizer. A little more buffer was added and the mixture stirred at 4° C. The supernatant was transferred to a dialysis tubing of 50 KDa exclusion limit (Spectrum Medical Instruments, USA) and dialysed overnight against PBS at 4° C. to remove the small ions. The tubulin solution was concentrated using ultrafiltration cones (Amicon) and kept in liquid nitrogen until required for analysis or immunization studies.

Synthetic Peptides

Two synthetic tubulin peptides (STh) corresponding to the carboxyl terminal of the β-tubulin CDNA (Kimel, B. et al. (1985) Gene, 35: 237-248) of T b. rhodesiense were ordered from the Sheldon Biotechnology Centre (McGill University, Canada). The STP 12 peptide with 12 amino acids (TEEEGEFDEEQY) was obtained already coupled to Key Hole Limpet Haemocyanin (KLH) whereas the STP 14 peptide with 14 amino acids (TIEEEGEFDEEEQY) was KLH-coupled in our laboratory using glutaraldehyde. Immuno-protection studies in mice

a) Immunization Studies

In order to determine whether immunization with tubulin would confer any protection and to establish a baseline for subsequent studies, an immunization and challenge experiment was performed. Briefly, mice were immunized subcutaneously with 40 μg and boosted with 20 kg and again with 20 μg of the native T. brucei tubulin or synthetic tubulin peptides at day 15 and day 30, respectively. For the initial immunizations, each of the antigens were added to an equal volume of Freund's complete adjuvant (FCA) and emulsified using a syringe and 22-gauge needle. Boosting was done using antigen emulsified in incomplete Freund's adjuvant (IFA). A control immunized with only adjuvant emulsified in PBS was similarly established. Mice were then challenged intraperitoneally with an otherwise lethal dose (10³ cells in 200 μl PSG) of the homologous strain of T. brucei (UTRO 0120291B). Parasitaemia was monitored daily for the first month and then every three days thereafter and the patent period (days post-challenge when parasites first appeared in tail blood) was determined for each mouse. The persistence span (days post-challenge when each infected mouse died) and the protection rate (percentage of mice which did not become patent and survived beyond 60 days post challenge) were also determined.

Further experiments were set up to (i) investigate the effect of the dose, adjuvant and route of immunization (Table 1A), (ii) compare immunizations using either native or denatured trypanosome tubulin, mammalian tubulin or synthetic tubulin peptides (Table 1B) and (iii) to study the response to heterologous challenge (Table IC). In each experiment, parasitaemia was monitored daily for the first month and every three days thereafter. The patent period, persistence span and the protection rate were determined.

TABLE 1
Immunization regimes
(a): Regime for determining the effects of dose,
adjuvant and route of
administration on efficacy of immunization
with native tubulin (nTbTub). Mice
were immunized subcutaneously or
intraperitoneally with native tubulin from
stock UTRO 01202291B with or without adjuvant
followed by subsequently
challenged with the homologous strain.

| | | Dose (mg) at day | | | |
|---|---|---|---|---|---|
| Antigen* | mice (n) | 0 | 15 | 30 | Route* |
| nTbTub | 15 | 40 | 20 | 20 | s/c |
| nTbTub | 15 | 40 | 20 | 20 | i/p |
| nTbTub | 10 | 20 | 20 | 20 | s/c |
| nTbTub No adj | 10 | 40 | 20 | 20 | s/c |
| Control | 10 | — | — | — | s/c |

| | |
|---|---|
| *Abreviations: | |
| nTbTub; | Native tubulin derived from T. brucei UTRO 020191B |
| nTbTub No Adj | nTub administered without adjuvant |
| Control | Adjuvant (Complete or incomplete Freund's adjuvant) |
| s/c; | Subcutaneous route of immunization |
| i/p; | Intraperitoneal route of immunization |

(b): Regime for comparison of native or
denatured trypanosome tubulin,
mammalian tubulin, and synthetic tubulin
peptides for immunization. Mice were
immunized subcutaneously with
the optimal dose of the antigen indicated and
subsequently challenged with the homologous strain.

| | | Dose (mg) at day | | |
|---|---|---|---|---|
| Antigen* | mice (n) | 0 | 15 | 30 |
| nTbTub | 15 | 40 | 20 | 20 |
| dTbTub | 10 | 40 | 20 | 20 |
| RbTub | 10 | 40 | 20 | 20 |
| STP14 | 10 | 100 | 50 | 50 |
| Control | 10 | — | — | — |

| | |
|---|---|
| *Abbreviations: | |
| nTbTub; | Native tubulin derived from T.brucei UTRO 020191B |
| dTbTub | Denatured TbTub recovered from SDS-PAGE gel |
| STP 14; | Synthetic tubulin peptide (STP14) |
| | based on T. rhodesiense |
| | b-tubulin c-DNA |
| TbTub; | Rat brain tubulin |
| Control | Adjuvant (Complete or incomplete Freund's adjuvant) |

(c): Regime for determining the efficacy of
immunization with native T. brucei
tubulin against homologous and
heterologous challenge. Mice were immunized
subcutaneously with the previously determined
optimal dose of native tubulin
from T. brucei stock UTRO 010291B and
challenged with a lethal homologous or
heterologous stock.
A lethal dose of T. brucei = (10³cells),
T. rhodesiense or T. congolense (both) = (10⁵cells).

| | | Dose (mg) at day | | | |
|---|---|---|---|---|---|
| Antigen* | mice (n) | 0 | 15 | 30 | Challenge stock^{#} |
| nTbTub | 15 | 40 | 20 | 20 | T.b 010291B |
| Control | 10 | — | — | — | T.b 010291B |
| nTbTub | 15 | 40 | 20 | 20 | T.b 220291D |
| Control | 10 | — | — | — | T.b 220291D |
| nTbTub | 15 | 40 | 20 | 20 | T.r 080291B |
| Control | 10 | — | — | — | T.r 080291B |
| nTbTub | 15 | 40 | 20 | 20 | T.c 161098B |
| Control | 10 | — | — | — | T.c 161098B |

| | |
|---|---|
| *Abbreviations: | |
| nTbTub; | Native tubulin derived from T. brucei UTRO 010291B |
| Control | Immunized with adjuvant (Complete or |
| | incomplete Freund's adjuvant) |
| T.b; | Trypanosoma brucei |
| T.c; | Trypanosoma congolense |
| T.r; | Trypanosoma rhodesiense |

^{#}The trypanosome stocks are denoted with a UTRO number

b) Passive Transfer of Immune Sera

Immune sera, 100 μl, from the protected group or pre-immunization sera from the control (unimmunized mice) were administered intravenously into naive irradiated mice. The mice were then challenged with a lethal dose of trypanosomes after 1 hr and monitored for protection as described above.

c) Sub-Inoculation of Mice with Brains from the Protected Mice

In order to establish whether the protected mice were sterile (completely free of trypanosomes) mice that did not show parasitaemia by day 60 were sacrificed and the brains dissected out and washed twice in PSG. They were cut into small pieces with a scalpel and teased out and centrifuged at 3,000 g for 10 minutes and the pellet resuspended in PSG. The 200 μl of this suspension was administered intraperitoneally into naive irradiated mice and the mice monitored for parasitaemia

d) Evaluation of the Level and Specificity of Antibody Responses

In order to determine the rate of development of the antibody response, following the subcutaneous and intraperitoneal route of immunization, blood was collected from the retro-orbital sinuses of mice at day 14, 21, 28 and 35 post immunization and the serum obtained and stored at −20° C. until used in ELISA and Western blot assays to study the level and specificity of the antibodies generated. Similarly, the development of the antibody responses to STP14 and nThTub were compared over time. Peak antibody responses for all the antigens were also compared at day 35 post-imrnunization.

The poly-L-lysine based ELISA (Lubega, G. W. and Prichard, R. K. (1991) Haemonchus contortus. Molecular and Biochemical Parasitology, 47: 129-138) was used to compare the antibody levels. For ELISA, each anti-serum was measured against nThTub, dThTub, RbTub or STP14 as antigen.

The Western blot was performed as described above to demonstrate clearly the specificity of the antisera produced in mice. For Western blot, total soluble extracts of T. brucei UTRO 010291B, T. rhodesiense UTRO 080291B, T. congolense UTRO 161098B and rat brain were run on 10% SDS-PAGE gel and transferred to nitrocellulose membrane and probed with the antisera against nThTub, or dTbTub (both derived from UTRO 010291B), or STP14 or RbTub derived from rat brain

f) Statistical Analysis

Data on protection were presented as means±standard errors of the mean (SEM). Significant differences (p-values) were determined by comparison of means by Student's t test or analysis of variance (ANOVA) or comparison of proportions where applicable.

Trypanosome Viability Studies Against Rabbit Immune Serum in Culture

a) Rabbit Immune Serum

In order to obtain sufficient amounts of serum for in vitro inhibition studies, parallel immunizations were performed in rabbits. About 100 μg of T. brucei or rat brain tubulin or synthetic peptide were solubilized in PBS pH 7.4 in a total volume of 0.5 ml and emulsified in an equal volume of Freund's complete adjuvant. Blood for the preparation of pre-immunization serum was drawn from the marginal ear vein after which the uniform emulsion (1 ml) was injected intradermally into one rabbit at multiple sites. After two weeks each rabbit was boosted with 50 μg of the same antigen emulsified in Freund's incomplete adjuvant. A second boost was performed in a further two weeks. Blood for preparation of the immune serum was drawn from the marginal ear vein, seven days after the last boost. The serum was diluted with an equal part of 5% (w/v) BSA in PBS pH 7.4 and stored at −20° C. until needed. The production and specificity of the antibodies in rabbit serum were determined using ELISA and Western blot as described above, before being used for trypanosome viability studies in culture.

b) Trypanosome Inhibition Assay in Culture

The assay was run in a 96-well plate. For short term assays (24 h) a high seeding density (2×10⁵ cells per ml) was applied, while for the long term assays (4-10 days), a low density (4×10³ cells per ml) was applied. The immune serum (containing an equal volume of 5% BSA) was diluted with an equal volume of complete bloodstream-form trypomastigote medium (CBM) (Baltz, T. et al. (1985) EMBO Journal 4: 1273-1277) and 75 μl of it added in duplicate into wells of column 11 of a 96-well tissue culture plate (T?P, Switzerland). CBM (50141) was then added to all the wells to be used in columns 2 to 10. Serial dilutions were then begun by transferring 25 μl from the appropriate wells of column 11 serially down to column 4. The 25 μl drawn from column 4 were discarded leaving wells of column 2 and 3 as control.

Pre-immunization serum was run in parallel with each immune serum. A suspension of trypanosomes previously culture-adapted by continuous growth in culture for at least 3 weeks was then diluted with CBM to give the required cell density per ml and 50 μl of it added into each well already containing the test or control samples. Wells of column 1 and 12 were not inoculated with trypanosomes but were filled with blank CBM to guard against evaporation from the outermost assay wells.

The plates were incubated at 37° C. under, 5% CO₂ and observed under an inverted microscope for growth characteristics and numbers every 24 hrs. For the long term assay, the medium was changed every 48 hrs, care being taken not to remove the trypanosome cells at the bottom of the wells. Trypanosome cells in each well were counted every 24 hrs using an improved Neumbeur haemacytometer.

c) Immuno-Agglutination Test

In order to determine whether antibodies could be involved in the mechanism of growth inhibition in vitro, an immuno-agglutination test was performed using the various immune sera and pre-immunization serum as control. Serum diluted with an equal part of 5% (w/v) BSA in PBS (pH 7.4) was added to a culture of trypanosomes in the log phase of growth in a 24-well culture plate and incubated for 30 min at 37° C. under 5% CO₂ and observed for agglutination under a microscope.

d) Immunofluorescence Test

To determine whether the antibodies were recognizing a surface or internal antigen, immunofluorescence tests were performed in two ways. The first test was performed on a suspension of intact trypanosomes in 1.5 ml microfuge tubes. The trypanosomes were treated with 1% (v/v) formaldehyde in PBS (pH 7.4) at 4° C. for 10 min, followed by washing (3-5 min) with PBS-G (0.1% (w/v) glucose in PBS) and centrifugation at 3000 rpm, 4° C. for 5 min. The pellet was incubated with 10% (w/v) fetal calf serum in PBS for 15 min to block non-specific antibody binding. After washing and centrifugation, the pellets were resuspended in PBS-G and equal volumes of antisera added and incubated for 1 hr at 25° C. After washing the pellets were incubated with diluted fluorescein-conjugated protein A and washed again. The pellets were then seeded onto a glass slide and observed under a fluorescence microscope.

The second test was performed on fixed permeabilised trypanosomes. Here the trypanosomes were smeared onto glass slides and air dried, followed by fixing for 10 min. with acetone-methanol mixture, 1:1 (v/v) pre-cooled to −20° C. The slides were washed with PBS to rehydrate the cells. The trypanosomes were permeabilised using 1% (v/v) Triton™-X 100 in PBS. Blocking and incubation with sera and conjugated protein A were performed as for the intact trypanosomes. All washing of slides were done using PBS-G on a rocking shaker.

Results

Purification of Tubulin

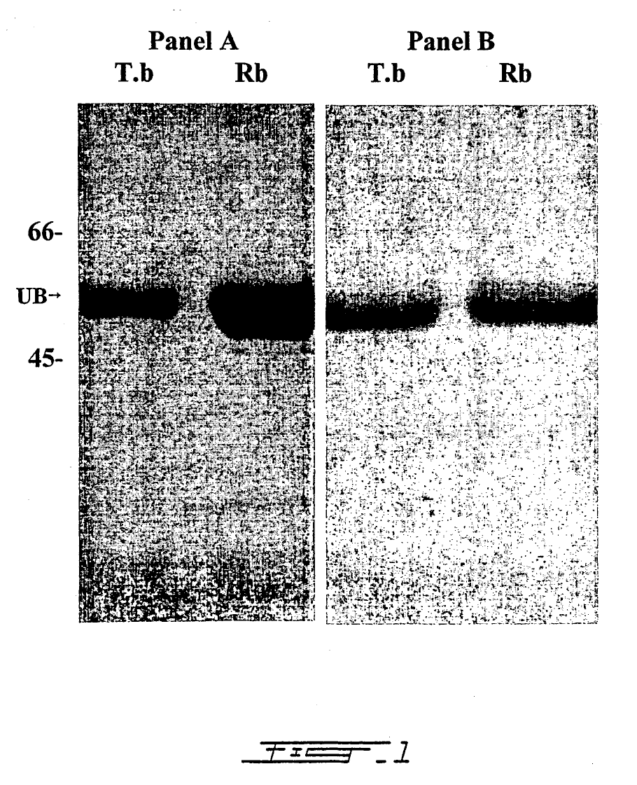

Tubulin was purified to near homogeneity and only one band corresponding to tubulin at 55 KDa was visible on SDS-PAGE gel stained with Coomassie blue (FIG. 1). To confirm that this band was tubulin a similar gel was run and transferred to a nitrocellulose membrane and probed with anti-chicken tubulin monoclonal antibody. The monoclonal antibody reacted strongly with the trypanosome tubulin and the rat brain tubulin at around 55 IDa (FIG. 1).

Immunoprotection Studies

(a) Investigation of Protection by Native T. brucei Tubulin and Tubulin Subunit Peptides in Mice

Native T. brucei tubulin (nThTub) was used to immunize mice which were subsequently challenged with the homologous strain of T. brucei. A total dose of 80 μg (nThTub) administered in 3 phases as described in Materials and Methods, was able to confer 100% protection to mice of which 67% (n=6) were completely protected (did not become patent at all) whereas the remaining 33% were partially protected since their patent period and persistence span were higher (p<0.05) than the controls (Table 2). A similar or even higher dose of synthetic peptides (STP12 or 14) did not confer any protection. Since there were no differences between the two peptides (STP12 and STP14) and only one of these peptides was used in subsequent experiments.

TABLE 2
Effects of immunization with native T. brucei tubulin and synethetic
peptides derived from T. brucei rhodesiense tubulin
Mice were immunized subcutaneously with the native T. brucei tubulin
(nTbTub), synthetic peptides (STP12 or 14), or with adjuvant alone
emulsified in PBS (control). All the mice were challenged with the
strain (UTRO 010291B) homologous to the nTbTub. The patent period
(number of days post-challenge when parasites first appeared in
tail blood) was determined for each mouse and the mean calculated
for the patent mice. The persistence span (mean number of days
post-challenge when the patent mice died) and the protection rate
(percentage of mice surviving beyond 60 days post challenge) were
also determined.

| | | | Mean | |
|---|---|---|---|---|
| | Patent | Mean Patent | Persistence | Protection |
| Antigen | rate (%)^{a} | period ± SEM^{b} | Span ± SEM^{c} | Rate (%)^{d} |
| Control | 100 | 4.6 ± 0.5 | 7.5 ± 0.5 | 0 |
| STP12 | 100 | 4.1 ± 0.4 | 6.5 ± 1.0 | 0 |
| STP14 | 100 | 4.3 ± 0.8 | 7.1 ± 0.8 | 0 |
| nTbTub | 33* | 6.0 ± 0.5** | 13.2 ± 1.5** | 67** |

^{a}Patent rate: percentage of mice in which parasites were detected in the tail blood
^{b}Mean patent period: mean number of days post-infection parasites were first detected in blood of the mice that became patent.
^{c}Mean persistence span; mean number of days survived by mice that became patent.
^{d}Protection rate; percentage of animals that did not become patent and survived beyond 60 days.
*Value significantly lower (p < 0.05) than control.
**Value significantly higher (p < 0.05) than control.

b) Effect of nTbTub Dose and Adjuvant on Protection

Of the mice immunized using the 40, 20, 20 kg regime by the subcutaneous route, 40% (n=15) developed parasitaemia but were partially protected since their mean patent period and persistence span were significantly higher (p<0.05) than the control (Table 3). The rest (60%) did not develop parasitaemia at all and were completely protected from the challenge. Sub-inoculation of their brains into naive irradiated mice showed that they were completely parasite free. However, when the immunizing dose was halved (20, 10, log regime) all the mice (100%) developed parasitaemia and died, although their persistence span and patent rates were significantly higher (p<0.05) than the controls. Therefore, the 40, 20, 20 μg regime was considered to be the minimum effective dose. It should be noted that all the mice immunized with this dose but without adjuvant, developed parasitaemia and died with no significant differences from the controls (p>0.05) in their patency rate and persistence span.

TABLE 3
Effect of nTbTub dose or adjuvant on protection
Mice were immunized subcutaneously with the nTbTub at a total dose of
80 μg (administered as 40, 20, 20 μg) or 40 μg (administered as
20, 10, 10 μg) in adjuvant or 80 μg but without adjuvant and a
control (mice injected with adjuvant emulsified in PBS) included. All
the mice were challenged with the strain (UTRO 010291B) homologous to
nTbTub. The patent period (number of days post-challenge when parasites
first appeared in tail blood) was determined for each mouse and the mean
calculated for the mice that became patent. The persistence span (mean
number of days post-challenge when the patent mice died) and the
protection rate (percentage of mice surviving beyond 60 days post
challenge) were also determined.

| | | | Mean | |
|---|---|---|---|---|
| Amount of | Patent | Mean Patent | Persistence | Protection |
| Antigen | rate (%)^{a} | period ± SEM^{b} | Span ± SEM^{c} | Rate (%)^{d} |
| Control (0 μg) | 100 | 3.6 ± 0.5 | 6.8 ± 0.8 | 0 |
| 40 μg | 100 | 6.8 ± 0.5** | 12.8 ± 1.1** | 0 |
| (in Adjv.). | | | | |
| 80 μg | 40* | 6.0 ± 0.5** | 13.2 ± 1.5** | 60** |
| (in Adjv.) | | | | |
| 80 μg | 100 | 3.5 ± 0.2 | 7.0 ± 0.5 | 0 |
| (no Adjv.) | | | | |

^{a}Patent rate: percentage of mice in which parasites were detected in the tail blood.
^{b}Mean patent period: mean number of days post-infection parasites were first detected in blood of the mice that became patent.
^{c}Mean persistence span: mean number of days survived by mice that became patent.
^{d}Protection rate: percentage of animals that did not become patent and survived beyond 60 days post infection.
*Value significantly lower (p < 0.05) than control.
**Value significantly higher (p < 0.05) than control.

c) Effect of Route of Immunization

Only 27% or 40% (n=15) of the mice became patent following immunization with nThTub via the intraperitoneal or subcutaneous routes, respectively. The remaining 73% and 60%, respectively, did not become patent and were completely protected from infection beyond 60 days post challenge (Table 4). The mice which became patent were partially protected since they became patent later and survived longer (p<0.05) than the control mice. Based on these parameters (patency rate, persistence span and protection rate) there was no significant difference (p>0.05) between the intraperitoneal and subcutaneous routes of immunization and therefore the subcutaneous route was used in subsequent experiments.

TABLE 4
Effect of route of immunization on protection
Mice were immunized with nTbTub (80 μg) either subcutaneously (SC) or
intraperitoneally (IP) and challenged with the strain (UTRO 010291B)
homologous to nTbTub. The control were inoculated with adjuvant
emulsified in PBS. The patent period (number of days post-challenge
when parasites first appeared in tail blood) was determined for each mouse
and the mean calulated for the mice that became patent. The persistence
span (mean number of days post-challenge when the patent mice died) and
the protection rate (percentage of mice surviving beyond 60 days post
challenge) were also determined.

| | | | Mean | |
|---|---|---|---|---|
| | Patent | Mean Patent | Persistence | Protection |
| Antigen | rate (%)^{a} | period ± SEM^{b} | Span ± SEM^{c} | Rate (%)^{d} |
| Control | 100 | 3.6 ± 0.5 | 6.8 ± 0.8 | 0 |
| SC | 40* | 6.0 ± 0.5** | 13.2 ± 1.2** | 60** |
| IP | 27* | 7.3 ± 0.9** | 16.5 ± 1.0** | 73** |

^{a}Patent rate: percentage of mice in which parasites were detected in the tail blood.
^{b}Mean patent period: mean number of days, post-infection, parasites were first detected in blood of the mice that became patent.
^{c}Mean persistence span: mean number of days survived by mice that became patent.
^{d}Protection rate: percentage of animals that did not become patent and survived beyond 60 days post infection.
*Value significantly lower (p < 0.05) than control.
**Value significantly higher (p < 0.05) than control.

d) Comparison of the Protection Due to nThTub, dbTub, RbTub and STP Antigens

Mice immunized with dThTub, RbTub or STP14 were not protected since there was no significant difference (p>0.05) in patent period, persistence span, or the protection rate between any of these groups and the control (Table 5). However for the mice immunized with nTbTub, 60% were completely protected and did not become patent throughout the experiment. The remaining 40% were partially protected since their patent and persistence periods were significantly higher (p<0.05) than the controls.

TABLE 5
Comparison of native and denatured T. brucei tubulin, mammalian tubulin
and a synthetic tubulin peptide Mice were immunized subcutaneously
with the native (nTbTub), or denatured trypanosome tubulin derived from
SDS-PAGE gel (dTbTub), or native tubulin from rat brain (RbTub),
synthetic peptide (STP14), or a control (adjuvant emulsified in PBS).
All the mice were challenged with the homologous strain, Trypanosoma
brucei (UTRO 010291B). The patent period (number of days post-
challenge when parasites first appeared in tail blood) was determined
for each mouse and the mean calculated for the mice that became patent.
The persistence span (mean number of days post-challenge when the
patent mice died) and the protection rate (percentage of mice surviving
beyond 60 days post challenge) were also determined.

| | | | Mean | |
|---|---|---|---|---|
| | Patent | Mean Patent | Persistence | Protection |
| Antigen | rate (%)^{a} | period ± SEM^{b} | Span ± SEM^{c} | Rate (%)^{d} |
| Control | 100 | 3.6 ± 0.5 | 6.8 ± 0.8 | 0 |
| STP14 | 100 | 3.7 ± 0.6 | 6.8 ± 0.7 | 0 |
| RbTub | 100 | 3.4 ± 0.5 | 7.2 ± 1.0 | 0 |
| dTbTub | 100 | 3.5 ± 0.2 | 6.9 ± 0.2 | 0 |
| nTbTub | 40* | 6.0 ± 0.5** | 13.2 ± 1.5** | 60** |

^{a}Patent rate: percentage of mice in which parasites were detected in the tail blood.
^{b}Mean patent period: mean number of days post-infection parasites were first detected in blood of the mice that became patent.
^{c}Mean persistence span: mean number of days survived by mice that became patent.
^{d}Protection rate: percentage of animals that did not become patent and survived beyond 60 days post infection.
*Value significantly lower (p < 0.05) than control.
**Value significantly higher (p < 0.05) than control.

e) Protection Against Heterologous Challenge

Mice were immunized subcutaneously with the minimum effective dose of native trypanosome tubulin (nThTub) and challenged with either T. congolense or T. rhodesiense or a different strain of T. brucei. There was protection observed against all of these strains of trypanosomes. In the group of mice challenged with a strain of T. brucei different from the one from which the immunogen was derived, only 36% (n=15) of the mice developed parasitaemia while 64% were completely protected and never developed any parasitaemia. For the 36% that developed parasitaemia, their patency period and persistence span were significantly higher than the control (p<0.05). In the groups challenged with T. congolense and T. rhodesiense, 73% (n=15) in each group were completely protected and never developed any parasitaemia (Table 6). The remaining 27% developed parasitaemia in each group but their patency period and persistence span were higher (p<0.05) than their controls. There was no significant difference (p>0.05) in the level of protection between the groups challenged with different species or strain.

TABLE 6
Protection against heterologous challenge
Mice were subcutaneously immunized with native T. brucei tubulin
(nTbTub) derived from strain UTRO 010291B and challenged with a
heterologous strain of T. brucei (UTRO 220291D) or with T. rhodesiense
(UTRO 080291B) or T. congolense (UTRO 161098B).
A control injected with adjuvant emulsified in PBS was included. The
patent period (number of days post-challenge when parasites first appeared
in tail blood) was determined for each mouse and the mean calculated for
the mice that became patent. The persistence span (mean number of days
post-challenge when the patent mice died) and the protection rate
(percentage of mice surviving beyond 60 days post challenge) were
also determined.

| | | | | Mean | Protection |
|---|---|---|---|---|---|
| | | Patent | Mean Patent | Persistence | rate |
| Challenge stock | Antigen | rate (%)^{a} | period ± SEM^{b} | Span ± SEM^{c} | (%)^{d} |
| T. brucei | Control | 100 | 3.6 ± 0.5 | 6.8 ± 0.8 | 0 |
| (UTRO 010291B) | nTbTub | 40* | 6.0 ± 0.5** | 13.2 ± 1.5** | 60** |
| T. brucei | Control | 100 | 6.5 ± 0.4 | 9.8 ± 1.8 | 0 |
| (UTRO 220291D) | nTbTub | 36* | 8.6 ± 0.7** | 17.6 ± 0.8** | 64** |
| T. rhodesiense | Control | 100 | 10 ± 0.4 | 36.0 ± 4.3 | 0 |
| (UTRO080291B) | nTbTub | 27* | 12.2 ± 0.6** | 46.4 ± 5.4** | 73** |
| T. congolense | Control | 100 | 5.0 ± 0.3 | 32.6 ± 4.0 | 0 |
| (UTRO161098B) | nTbTub | 27* | 9.8 ± 0.9** | 45.5 ± 2.9** | 73** |

^{a}Patent rate: percentage of mice in which parasites were detected in the tail blood.
^{b}Mean patent period: mean number of days post-infection parasites were first detected in blood of the mice that became patent.
^{c}Mean persistence span: mean number of days survived by mice that became patent.
^{d}Protection rate: percentage of animals that did not become patent and survived beyond 60 days post infection.
*Value significantly lower (p < 0.05) than the value of the corresponding control for that strain.
**Value significantly higher (p < 0.05) than the value of the corresponding control for that strain.

f) Passive Transfer of Immunity

Five mice were passively given immune sera, and subsequently challenged with the homologous T. brucei UIRO 020191B strain. Only one (20%) developed parasitaemia but it died later than control mice who did not receive immune sera, but were similarly challenged. There was no parasitaemia detected in the other four (80%) mice given immune sera, after the 60 days of monitoring.

g) Rate of Antibody Development: Effect of Route of Immunization and Synthetic Tubulin Peptides

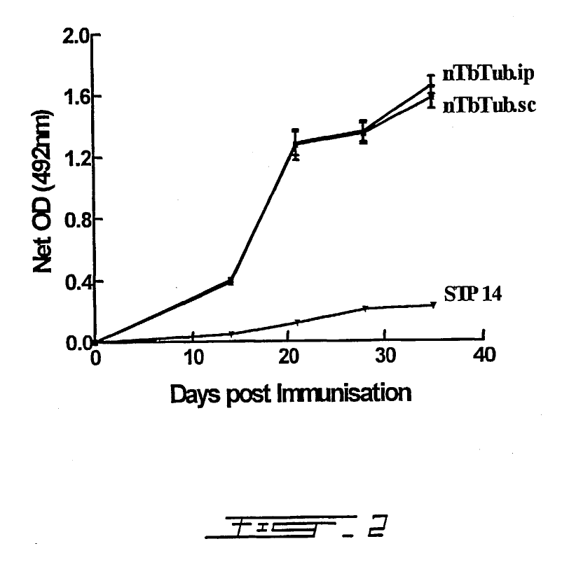

Mice were immunized with nThTub subcutaneously or intraperitoneally and the antibody responses over time determined by ELISA (FIG. 2). There were no significant differences (p>0.05) between the two routes of immunization in the level or rate of development of the antibody response. At the same time another group of mice was immunized subcutaneously with synthetic peptide (STI14) and the antibody responses over time compared with that due to nThTub. The level or rate of development of the antibody response due to STP14 was much lower than that due to nThTub and remained so throughout the experiment despite the various immunization boosts (FIG. 2).

h) Evaluation of Antibody Specificity by Western Blot

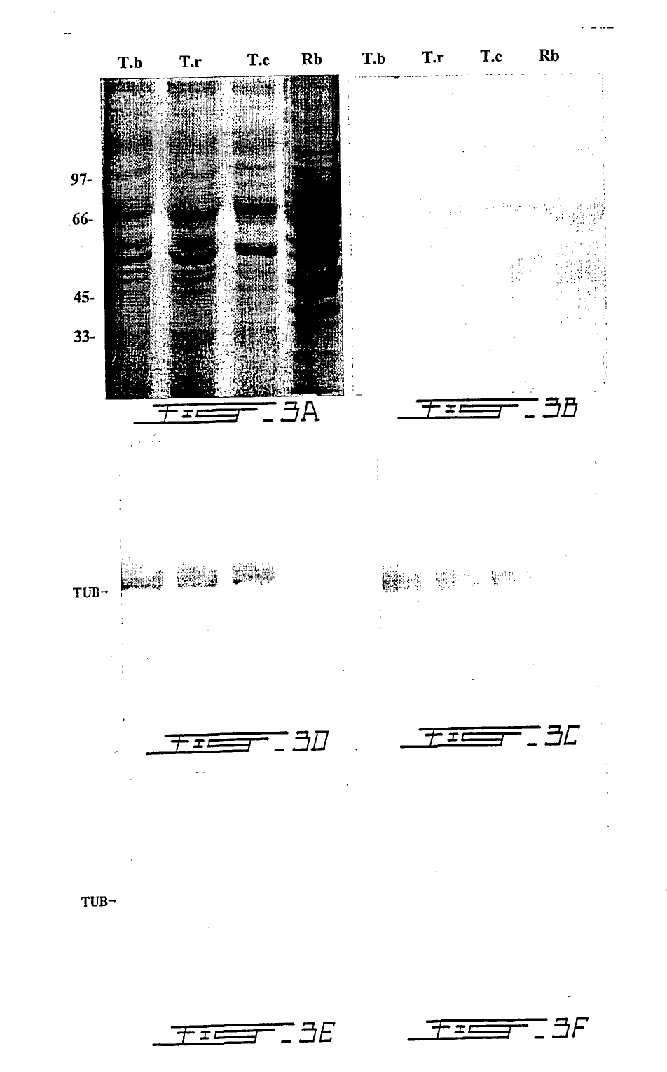

Coomassie staining of crude extracts from different trypanosome strains and rat brain following SDS-PAGE (FIG. 3, Panel A) revealed the presence of a variety of proteins in each extract. Antibodies raised against rat brain tubulin specifically recognized both mammalian and trypanosome tubulin of all the species and strains studied (FIG. 3, panel B). All the anti-trypanosome tubulin (whether anti-native, -denatured or -peptide) sera specifically recognized a single band corresponding to trypanosome tubulin on SDS-PAGE gel (FIG. 3), irrespective of species or strain, but did not recognize rat brain tubulin (FIG. 3, Panel C, D, and E). Pre-immunization sera failed to recognize any antigen (FIG. 3, Panel F).

i) Evaluation of the Antibody Levels and Specificity by ELISA

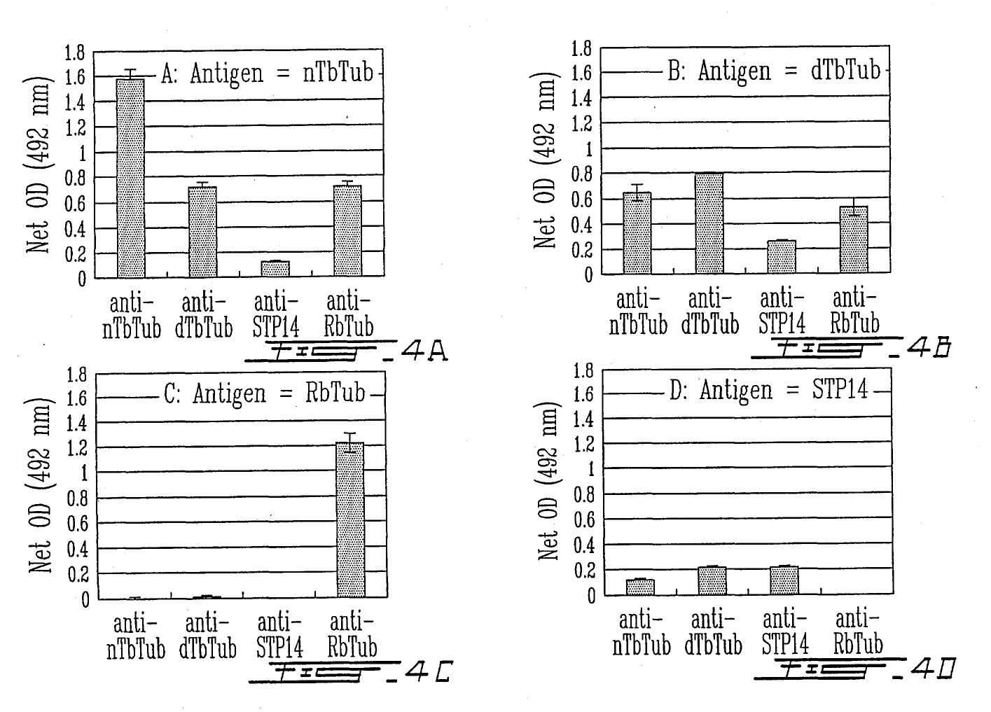

The various antisera raised against different antigens were compared by ELISA for specificity and antibody levels (FIG. 4). The specificity of the sera by ELISA was similar to that observed in Western blots. In particular, the anti-trypanosome sera (anti-nThTub, -dThTub and -ST 14) did not recognise rat brain tubulin but the anti-rat brain tubulin antibodies did recognise all the trypanosome tubulins, except the synthetic peptides, whereas the anti-nThTub and anti-dThTub did recognise the trypanosome tubulin including the peptides (FIG. 4). Although there were cross reactions, the amplitude of the reactions was strongest against self antigens in all cases.

In vitro Studies Using Sera From Immunized Rabbits

a) Specificity of the Rabbit Immune Serum

The specificity and antibody response patterns, using immune sera raised in rabbits against trypanosome tubulins, produced similar ELISA and Western blot results as those obtained with the corresponding mouse immune sera. For example, in Western blots, the rabbit anti-nThTub, anti-dTbTub and anti-STP serum specifically recognised trypanosome tubulin but not rat or chicken brain tubulins. However, the rabbit anti-rat tubulin serum recognised rat brain tubulin but not trypanosome tubulin.

b) Trypanosome Inhibition in Culture

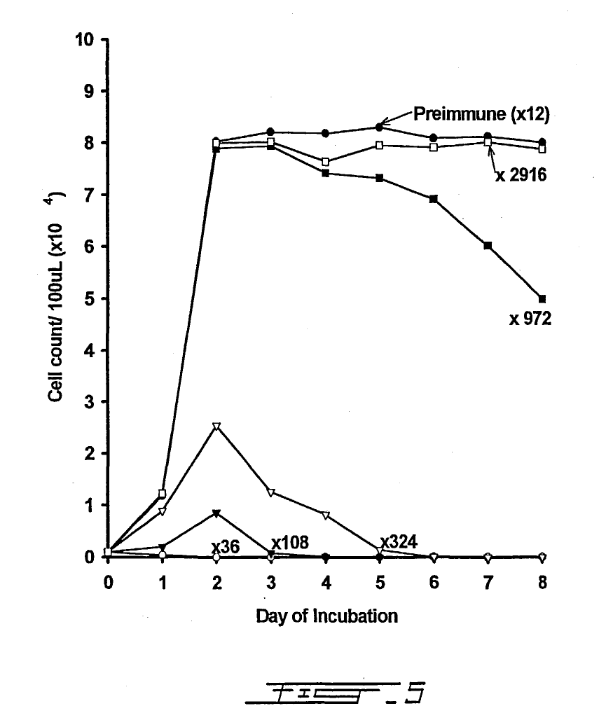

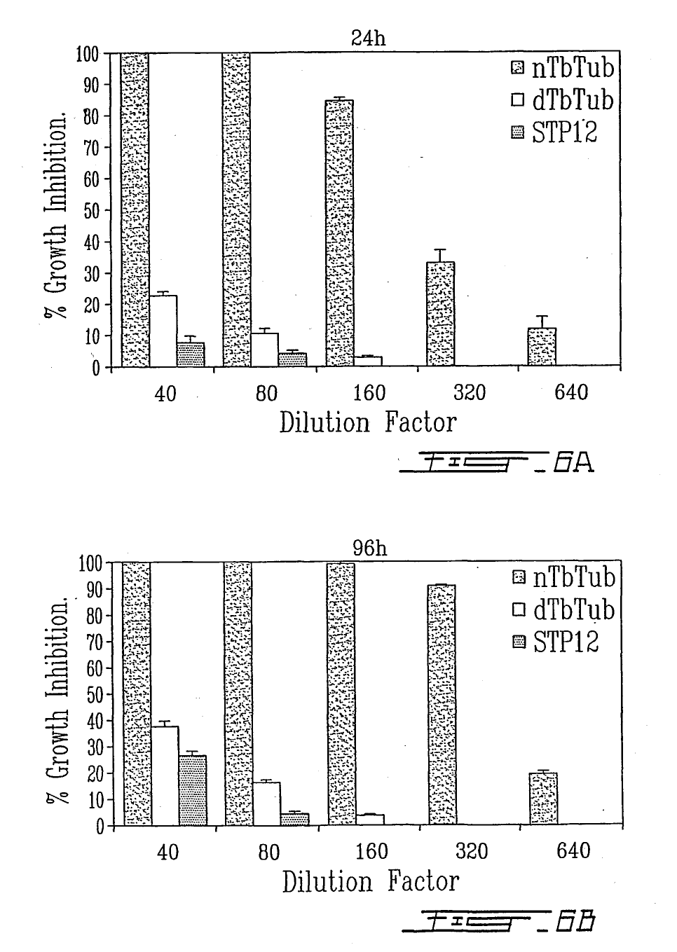

Pre-immunization rabbit serum diluted 10 times or more had no effect on trypanosome growth. Therefore, all test sera were diluted at least 10 times and compared with the pre-immunization serum diluted 12 times. The anti-nThTub serum strongly inhibited trypanosome growth in culture and the inhibition effect decreased with increasing dilutions but increased with incubation-time such that by day 8 even serum diluted by nearly ×1000 visibly reduced trypanosome growth. In contrast, the pre-immunization serum diluted 12 times did not affect trypanosome viability (FIG. 5). A similar pattern was observed for the anti-dTbTub, anti-STP12 or anti-STP14 sera in that the inhibition of trypanosome growth was affected in a dilution and incubation-time dependent manner as described above. However, the percentage inhibition for these anti-sera was less than for the anti-nThTub serum at the equivalent dilutions and incubation times (FIG. 6). The rank order of activity of the sera was: nThTub>>dThTub>STP12=STP14 and appeared to correlate with the rank order of the antibody levels observed by ELISA (see above).

c) Immunoagglutination test

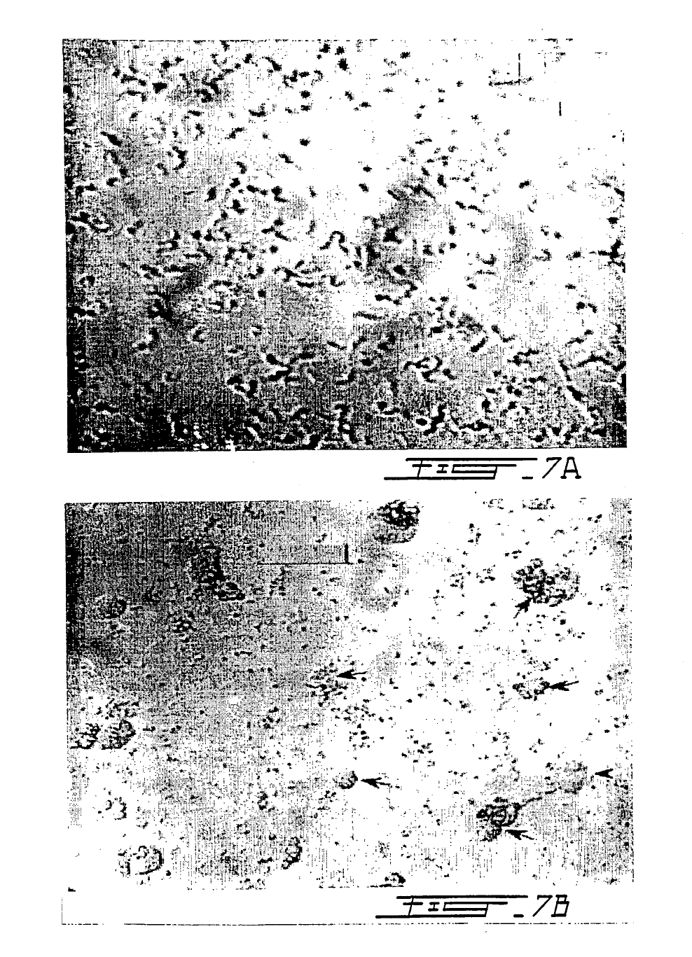

The pre-immunization serum did not cause any agglutination of trypanosomes within the 30 min incubation time but there was pronounced agglutination with the anti-nThTub serum (FIG. 7). Even the free (non-agglutinated) trypanosomes in the culture incubated in the anti-nThTub serum were markedly deformed. Agglutination also occurred with the anti-dThTub and anti-STP sera but not with anti-rat brain tubulin. In addition, treatment with heat (56° C.), which inactivates complement, did not eliminate the agglutinating effect, where it occurred.

d) Immunofluorescence Test

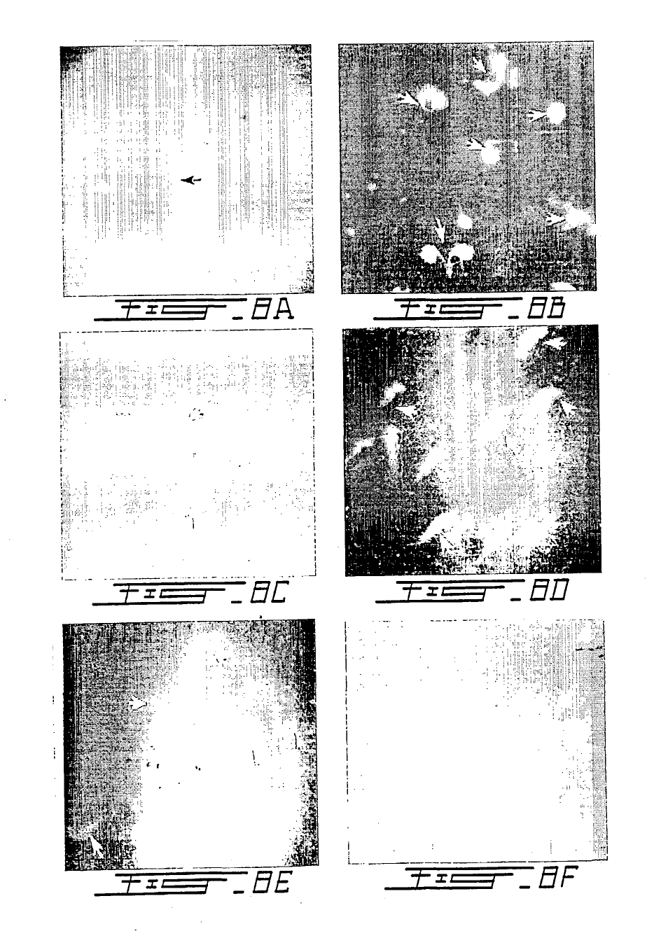

There was intense and uniform fluorescence when the fixed permeablised trypanosomes were probed with the anti-nThTub, anti-dThTub or anti-STP12, but there was no fluorescence when anti-rat tubulin or pre-immunization sera were used (FIG. 8). However, when the intact trypanosomes were probed with any of the sera, except the anti-RbTub and the pre-immunization sera, there was fluorescence only in a small area near the posterior end of the trypanosomes.

Discussion

This study was carried out to explore whether tubulin would be a target for a vaccine against trypanosomiasis. In particular it was to investigate (i) whether immunization of mice with tubulin or synthetic peptides of tubulin could confer any protection, (ii) to determine the conditions under which such protection would occur and (iii) to establish whether rabbit anti-tubulin or anti-tubulin peptide sera would alter the viability of trypanosomes in culture.

Tubulin was purified from one strain of T. brucei and analyzed for purity before being used to immunize mice or rabbits. Only a single band around 55 KDa was observed, after staining with Coomassie blue, which corresponded with tubulin purified from rat brain. Both were recognized by commercial anti-chicken tubulin monoclonal antibodies (Amersham) in Western blots (FIG. 1).

In the experiments in which mice were immunized with the native T brucei tubulin and challenged with a lethal dose of trypanosomes, between 60 and 80% (n=81) were completely protected without becoming patent, whereas the remaining mice were partially protected with longer patency and persistence spans than the controls. Tubulin from T. brucei (UTRO 010291B) not only protected against challenge with a homologous strain of T. brucei but also against challenge with a heterologous strain of T. brucei or T. congolense or T. rhodesiense (Tables 4-8). This is interesting because it suggests that the variable surface glycoprotein (VSG) was not responsible for the immunoprotection observed The VSG only protects against the homologous unpassaged challenge (Scott et al., 1978). This means that trypanosome tubulin from one strain can confer protection against many strains and species. The mechanism by which the protection in this study occurred seems to have been antibody mediated since passive transfer of antibodies resulted in protection against challenge. We are the first to report that tubulin, the principal microtubule protein, can confer complete immunoprotection against trypanosomiasis.

Protection was reported against American trypanosomiasis (T. cruzi infection) using the purified paraflagella rod protein (Wrightsman, R A. et al. (1995) Infection and Immunity, 63:122-125) or whole flagella fraction quiz, A. M. et al. (1990) Molecular and Biochemical Parasitology, 39: 117-126). Use of the purified paraflagella rod protein did not prevent infection in any mice but the parasitaemia was reduced and all the mice survived up to 120 days (Wrightsman, R A. et al. (1995) Infection and Immunity, 63:122-125). However, the paraflagella rod protein has not been tried in African trypanosomiasis. Use of the whole flagella fraction against T. crui resulted in 60% of the mice being completely protected without any parasitaemia and the remaining 40% becoming partially protected, but immunization with the flagella pocket fraction against African trypanosomiasis resulted in partial protection only (Mkunza, F. et al. (1995) Vaccine, 13: 151-154). The other study in which complete protection was reported involved immunization with a fraction containing MAP⁵² and two glycosomal enzymes and resulted in 100% complete protection without any mouse becoming patent upon challenge with trypanosomes (B3alaban, N. et al. (1995) Journal of Infectious Diseases, 172: 845-850). It is interesting that both tubulin and these other protective fractions (flagella and MAP52) have some relationship with microtubules and the antibodies against these after protective antigens localized in the flagellum, the body of the parasite, the membrane, and the flagella pocket.

ELISA and Western blot studies showed that the antibodies against the T. brucei tubulin we used recognized tubulin of the other strains and species of trypanosomes studied (FIG. 3). It is therefore not surprising that mice were protected against challenge with heterologous strains of T. brucei and other species. Most of the subcellular antigens are non variable across species but their access to the host immune cells may be difficult because the VSG is not only abundant but very well exposed on the surface and is recognised at the expense of (out competes) the subcellular antigens. However, when these subcellular antigens are presented in pure form to the immune system, they can induce a strong and protective immune response. Therefore, whereas the immune recognition cells may not access the internal antigens, the antibodies they generate can be internalised, by a mechanism not yet established. It is possible that the flagella pocket can play a role in this internalisation process. It is known that antibodies play a significant role in controlling trypanosome infection and indeed, in this study, passive transfer of anti-tubulin serum to naive mice resulted in 80% complete protection, indicating that the protection observed was humoral. This was also confirmed by the serum inhibition studies whereby trypanosome proliferation in culture was specifically inhibited by the anti-tubulin antibodies (FIG. 5 & 6). It is suggested that parasites that replicate extracellularly, like the African trypanosomes, can be controlled by antibodies through one or more mechanisms. In one of these mechanisms, antibodies may bind these parasites and block their attachment to the host receptors and interfere with their entry and establishment in their predilection site. This can be the mechanism by which our mice that became protected never became patent. It is interesting that all the mice that became patent eventually died even though this took longer than the control, suggesting that the mice failed to clear the infection once it got established in the blood system. The reason for this is not clear since tubulin is incapable of antigenic variation However, it is possible that the antibody levels became depleted since the tubulin of the intact trypanosomes is unlikely be accessed by the host's immune recognition cells in order to boost the immune response. Alternatively, it may be related to the immunosuppressing ability of the established trypanosomes. On the other hand, the immune serum was able to inhibit trypanosome growth in culture where attachment receptors are not required. However, tubulin is involved in cell division via the mitotic spindle and other processes. It is possible that the antibodies blocked trypanosome cell division but other humoral-effector mechanisms such as agglutination, lysis and complement (Newman, M. J. et al. (1995) Immunological Formulation design considerations for subunit vaccines. In: M. F. Powell and M. J. Newman (ed) The Subunit and Adjuvant Approach. Plenum Press, New York) could have played a role in vivo. In this study the anti-trypanosome tubulin immune serum caused agglutination of trypanosomes in culture but non-immune serum or anti-rat brain tubulin serum did not (FIG. 7). However, lysis was not observed and this agglutination was not inactivated by pre-heating (56° C.) the serum suggesting that complement was not involved. Agglutination was probably caused, indirectly, by the internalized antibodies but not via cross-linking of surface bound antibodies since the immunoflorescence test did not reveal antibodies on the surface in this study or other studies (Balaban, N. et al. (1995) Journal of Infectious Diseases, 172: 845-850). It was proposed that stress due to the internalized antibodies can cause agglutination (Balaban, N. et al. (1995) Journal of Infectious Diseases, 172: 845-850). This study could therefore not conclusively establish the mechanism by which the antibodies caused the protection we observed.

Among the most interesting observations was that tubulin from one strain of T. brucei conferred protection against challenge from heterologous strains of the same or different species. Thus tubulin from one strain conferred a broad protection against African trypanosomiasis. Secondly, the anti-tubulin antibodies did not uniformly stain the surface of the trypanosomes in an immunofluorescence test (FIG. 8). They stained a small part of the posterior end which probably represents the region (flagella pocket) uncovered from the VSG coat. The heterologous protection and the antibody staining pattern conclusively rules out the involvement of the VSG in the protection.

β-tubulin rather than α-tubulin is primarily targeted by anti-tubulin drugs (Lubega, G. W. and Prichard, R. K. (1991) Haemonchus contortus. Molecular and Biochemical Parasitology, 47: 129-138). Therefore, in this study synthetic peptides from the most variable (i.e. unique for every organism) and immunogenic part of B-tubulin cDNA, the C-terminus (Kimmel, B. et al. (1985) Gene, 35: 237-248) were tested for their immunoprotection abilities. These peptides were not protective upon challenge, although they induced antibodies which recognised tubulin from all the trypanosomes tested but not mammalian tubulin (FIG. 3). It is possible that this failure was due to the fact that the peptides induced a much lower antibody level than the whole native tubulin (FIG. 4.). This was supported by a parallel study in culture whereby rabbit anti-peptide serum of exactly the same specificity, exhibited killing of trypanosomes but with much less effect than the anti-native tubulin antibodies at comparable dilutions (FIG. 6). Therefore a mechanism which can boost the levels of these anti-peptide antibodies in blood may render them protective. Alternatively, it is possible that these peptides, which contain only very short sequences from the C-terminal of β-tubulin, did not represent the protective epitopes. It would be interesting to know how the peptides from other regions of B-tubulin would behave and to substantiate the role of α-tubulin, if any. Alpha-tubulin might play a role, at least in the immune induction mechanisms, as it does to stabilise the benzimidazole (anthelmintic) binding site of nematode β-tubulin (Lubega, G. W. et al. (1993) Molecular and Biochemical Parasitology, 62: 281-292). These studies indicate that conformation might play a role in the immunogenicity of the protective epitope because whole tubulin isolated by SDS-PAGE (and denatured) induced a much lower level of antibody response (FIG. 4) and did not confer any protection to immunized mice on challenge. Again, the rabbit anti-T. brucei denatured tubulin antibodies exhibited killing of trypanosomes in culture but with much less effect than the anti-native antibodies at comparable dilutions (FIG. 6). Thus the level of antibodies (titre) and therefore the immunogenicity may have played a role in the tubulin immuno-protective ability. It would be interesting to express the ax and B-tubulin isoforms in a single and dimeric native state and use them in immunoprotective studies. This type of study is planned for the near future. A study involving overlapping peptides spread over the whole α or β-tubulin isoform is also planned in order to identify the protective epitope(s). Also required is a study to identify antigen-delivery mechanism or conditions that result in an optimal antibody response. A recombinant vaccine based on tubulin would be an interesting advancement for human and animal African trypanosomiasis control since 60-80% protection would result in a significant reduction in the transmission of trypanosomiasis. Mice immunized with the anti-rat brain tubulin died at the same time as the controls. Thus the protection was apparently specific to trypanosome derived tubulin; and despite tubulin being present in the host, immunization with trypanosome tubulin would probably not cause serious auto immune reactions. This is also supported by the fact that both rabbit and mouse anti-trypanosome tubulin antibodies recognised only tubulin in trypanosome but not mammalian soluble extracts.

We have presented for the first time, data which indicates that tubulin is a promising target for development of a parasite specific, broad-spectrum anti-African trypanosomiasis vaccine.

The present invention will be more readily understood by referring to the following example which is given to illustrate the invention rather than to limit its scope.

### EXAMPLE I

### Anti-Tubulin Antibody With Trypanocidal Activity

An experiment was set up to investigate whether a contaminating antigen is responsible for the immunoprotection by the native trypanosome tubulin (NTP). Previous data showed that whereas immunization with NTP was protective in mice, synthetic β-tubulin peptides (STP) from the variable and immunogenic C-terminal or denatured tubulin, purified by SDS-PAGE (dNTP) were not significantly protective in vivo. This raised the possibility interpretation that SDS-PAGE removed a contaminating antigen which could be responsible for the protection. However, it should be noted that all immune sera (anti-NIP, anti-STP or anti-dNTP) were trypanocidal when directly applied to trypanosomes in culture, although the anti-NTP was far more active (effective at much lower titre) than the anti-dNTP or anti-STP, and suggests that DNTP and STP may be immunogenic in vivo, but not sufficiently so to be protective.

In order to unequivocally demonstrate that antibody to trypanosome tubulin was the lethal component in the antisera raised against NIP or STP, anti-tubulin antibody was adsorbed using the SDS-PAGE purified denatured tubulin (dNTP) and the trypanocidal activity of the sera assessed.

Materials and Methods

Trypanosome tubulin was purified (NTP) and used to immunize rabbits as described previously. Synthetic peptides (STP) used previously were similarly used to raise immune serum. In both cases pre-immune and immune sera were collected and processed in the usual manner. The native trypanosome tubulin (NTP) was further purified by SDS-PAGE in order to remove any contaminating antigen. We previously described this preparation as denatured trypanosome (dNTP) and we used it in this experiment to determine if it would remove (adsorb) the anti-tubulin (NTP) or anti-STP antibodies and block their inhibition of trypanosome proliferation in culture.

Trypanosomes were cultured and the immune serum applied as previously described.

Results and Discussion

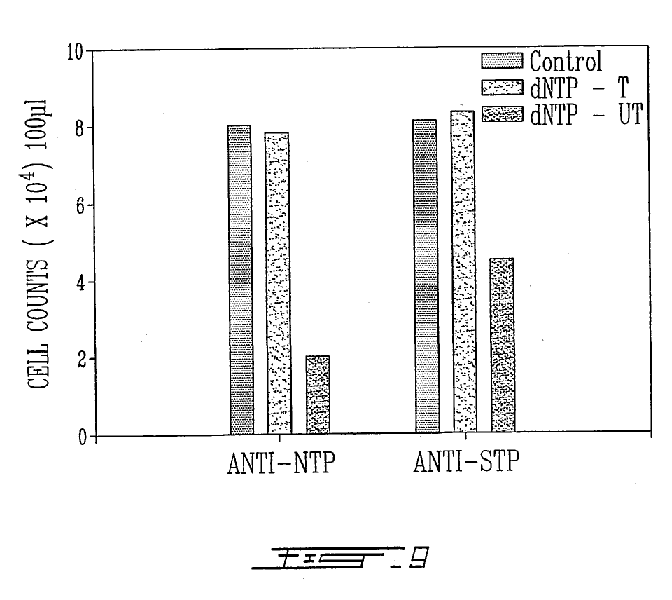

Treatment of the anti-NTP or anti-STP serum with dNTP completely abolished the trypanocidal activity of either serum (FIG. 9). The data indicates that the trypanocidal activity is due to anti-tubulin antibody and not an antibody to a contaminating antigen in the NTP.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A substantially pure tubulin preparation, which comprises a tubulin extract from Trypanosoma brucei, wherein said tubulin preparation can protect animals and humans against heterologous strains of different species of Trypanosoma.

2. A method for the immunization of an animal or a human patient against heterologous strains of different species of Trypanosoma, which comprises administering to said animal or human patient an immunogenic amount of a tubulin extract preparation isolated from a Trypanosoma.

3. The method of claim 2, wherein said Trypanosoma is Trypanosoma brucei.

4. A vaccine for immunizing animals or humans against trypanosomiasis, which comprises an immunogenic amount of a tubulin extract preparation isolated from a Trypanosoma or an immunoprotective amount of an antibody raised against a tubulin isolated from a Trypanosoma.

5. The vaccine of claim 4, wherein said Trypanosoma is Trypanosoma brucei.

6. An antibody raised against the tubulin preparation of claim 1.

7. The antibody of claim 6, which is a polyclonal or a monclonal antibody.

8. A vaccine for immunizing against trypanosomiasis in animals or humans, which comprises an immunogenic amount of a recombinant tubulin which corresponds in composition to a tubulin extract preparation isolated from a Trypanosoma or an immunoprotective amount of an antibody raised against said recombinant tubulin.

9. A vaccine for immunizing against trypanosomiasis in animals or humans, which comprises an immunogenic amount of a synthetic peptide which corresponds in composition to portion of an amino acid sequence of a tubulin extracted from a Trypanosoma or an immunoprotective amount of an antibody raised against said tubulin peptide.

10. Use of an immunogenic amount of a tubulin preparation of claim 1 for immunization of an animal or a human patient against heterologous strains of different species of Trypanosoma.

11. The use of claim 10, wherein said Trypanosoma is Trypanosoma brucei.
